(19) United States
(12) Patent Application Publication
(10) Pub. No.: US 2024/0374283 A1
(43) Pub. Date: Nov. 14, 2024

(54) NEEDLE AID DEVICE

(71) Applicant: VivaChek Biotech (Hangzhou) Co., Ltd., Zhejiang (CN)

(72) Inventors: Qianli MA, Zhejiang (CN); Feifei LI, Zhejiang (CN); Zhen MA, Zhejiang (CN); Zheyao YANG, Zhejiang (CN); Jianlin DING, Zhejiang (CN); Wenling ZHANG, Zhejiang (CN); Qinggang YANG, Zhejiang (CN)

(73) Assignee: VivaChek Biotech (Hangzhou) Co., Ltd., Zhejiang (CN)

(21) Appl. No.: 18/686,874

(22) PCT Filed: Aug. 24, 2022
(86) PCT No.: PCT/CN2022/114606

§ 371 Date: Feb. 27, 2024

(30) Foreign Application Priority Data
Aug. 27, 2021 (CN) 202110992416.6
Aug. 27, 2021 (CN) 202122036465.0

(51) Int. Cl. A61B 17/34

(52) U.S. Cl. CPC A61B 17/3403

(57) ABSTRACT

A needle aid device comprises an outer shell; an inner shell located in the outer shell and comprising an upper portion and a lower portion, a trigger shell passage penetrating through the upper portion and the lower portion being formed in the inner shell, and an inner diameter of the lower portion being greater than that of the upper portion; and a trigger module capable of moving in the trigger shell passage and used for clamping or releasing an emitter mechanism; wherein, in an initial state, the trigger module is locked on the upper portion; and when unlocked, the trigger module drives the emitter mechanism to move downward until the emitter mechanism is separated from the trigger module.

### TECHNICAL FIELD

The invention belongs to the technical field of continuous glucose monitoring, and particularly relates to a needle aid device.

### TECHNICAL BACKGROUND

A continuous glucose monitoring system (CGMS) is typically composed of a GOx-based needle-type electrochemical glucose sensor to be micro-invasively implanted into human skin, a wireless or wired signal detection and transmission/recording device (emitter), a processor for converting a detected current signal into a glucose concentration (generally, the processor is arranged in an App or a receiver), and a needle aid for implanting the sensor into skin. The sensor is implanted into the skin of a patient by means of the needle aid, an electric signal is generated when the sensor undergoes, in interstitial fluid, an oxidation reaction with glucose in the body of the patent, the electric signal is then converted into a glucose reading, and the glucose reading is transmitted by the emitter to the receiver. With the guidance of these data and illustrative diagrams, clinicians can gain a comprehensive understanding of glucose fluctuations of the patent all the time, and inject insulin to the patient with an insulin pump, if necessary.

The thickness of subcutaneous tissue varies drastically with the gender, body parts and body mass index. Infrared light which can penetrate through human tissue is often used to detect the thickness of human skin, and in some cases, ultrasonic detection waves and ultrasonic echo signals are used to detect the thickness of human skin.

The CGMS can correctly reflect the change of human blood glucose, which is on the basis of the hypothesis that the glucose concentration in interstitial fluid is extremely similar to the blood glucose concentration because the glucose in interstitial fluid in human blood capillaries is highly correlated with the blood glucose, so current produced by a biochemical reaction of the glucose in interstitial fluid can be converted into a detection value of the blood glucose, with reference to the calibration of the blood glucose level, which means that the accuracy of the sensor will be higher if the sensor is closer to a body part with abundant blood capillaries. The CGMS is often implanted into the subcutaneous fat layer as required, and for different human bodies, there are differences in the thickness of the fat layer and the abundance of the blood capillaries due to different physical conditions. There are two types of human fat, which are white fat and brown fat, wherein the white fat is accumulated under the skin and is responsible for storing redundant energy, making human bodies become fatter; brown fat cells contain a large quantity of mitochondria and are rich in blood capillaries, so it is recommended that the sensor of the CGMS is implanted into human body parts with brown fat or the joint of white fat and brown fat. By adjusting the implantation depth of the sensor, the maximum effective area of the sensor can be obtained, guaranteeing the sensitivity and accuracy of the sensor.

Before being implanted, the sensor probe is placed in a semi-closed needle, and under the action of the needle aid, the needle can be implanted under skin together with the sensor; and the needle can be quickly withdrawn from human skin, while the sensor is left under the skin. Therefore, it is necessary to provide a needle aid to realize needle implantation and withdrawal quickly and stably.

Existing needle aids have a complex structure and cannot withdraw needles stably, which compromises the stability of the sensor probe implanted under the skin. In order to realize accurate implantation of the sensor, the needle aid should make the guide needle adjustable.

### SUMMARY OF THE INVENTION

To overcome the defects in the prior art, the invention provides a needle aid device which can be triggered more easily and can implant and withdraw needles stably.

The technical solution adopted by the invention to solve the technical problems is as follows: a needle aid device comprises:
an outer shell;
an inner shell located in the outer shell and comprising an upper portion and a lower portion, a trigger shell passage penetrating through the upper portion and the lower portion being formed in the inner shell, and an inner diameter of the lower portion being greater than that of the upper portion; and
a trigger module capable of moving in the trigger shell passage and used for clamping or releasing an emitter mechanism;
in an initial state, the trigger module being locked on the upper portion; and
when unlocked, the trigger module drives the emitter mechanism to move downward until the emitter mechanism is separated from the trigger module.

In the invention, the inner shell comprises the upper portion and the lower portion, which have different inner diameters, such that the trigger module can be locked and can be switched from the locked state to an unlocked state easily, that is, the needle aid device is easier to trigger; moreover, the trigger module and the inner shell can cooperatively move more stably.

Further, elastic clamping jaws are formed on the trigger module and are used for clamping the emitter mechanism, a thin-walled portion is formed on the lower portion of the inner shell, and when moving downward to the thin-walled portion, the elastic clamping jaws release the emitter mechanism. The thin-walled portion is simple and ingenious in structural design, and automatic unlocking can be realized when the elastic clamping jaws move to the thin-walled portion.

Further, the trigger module is provided with a trigger buckle; when the trigger module is locked, the trigger buckle is clamped at an opening of the trigger shell passage; and when an external force is applied to the trigger button to push the trigger buckle away from a top surface of the inner shell, the trigger module is unlocked.

Further, a trigger button and a trigger button connecting terminal are connected to the outer shell, the trigger button is movably connected to the trigger button connecting terminal, and an external force is applied to the trigger button to unlock the trigger module. The trigger module is easier to unlock by means of the trigger button outside the outer shell.

Further, the trigger module further comprises a lock bracket, the lock bracket comprises a clamp and a collet, and the clamp clamps the trigger button to prevent the trigger button from moving with respect to the outer shell. The design of the lock bracket and the trigger button prevents mis-trigger of users, thus improving the safety of the needle aid device in use.

Further, trigger shell guide ribs are arranged in the trigger shell passage, the trigger module is provided with guide grooves, and the trigger shell guide ribs are clamped in the guide grooves and allowed to move in the guide grooves to realize vertical movement of the trigger module. Through the cooperation of the trigger shell guide ribs and the guide grooves, the whole needle implantation process is steadier, and the trigger module can horizontally move more stably.

Further, trigger shell guide grooves are formed in an inner wall of the inner shell, the trigger module is provided with bottom guide grooves, and the trigger shell guide grooves are matched with the bottom guide grooves and allowed to move with respect to the bottom guide grooves to realize vertical movement of the trigger module. Through the cooperation of the trigger shell guide grooves and the bottom guide grooves, the whole needle implantation process is steadier, and the trigger module can horizontally move more stably.

Further, the needle aid device further comprises a guide needle passage formed in the inner shell and a needle withdrawing member for clamping or releasing a guide needle mechanism, and a cavity allowing a sensor of the emitter mechanism to stretch therein is formed in a guide needle.

Further, the needle aid device further comprises an adjusting knob passage formed in the inner shell, an adjusting knob, an adjusting knob connecting terminal, an adjustable guide needle mechanism, and a needle withdrawing member for clamping or releasing the adjustable guide needle mechanism; part of the adjusting knob stretches into the adjusting knob passage, the adjusting knob is rotatably connected to the adjusting knob connecting terminal, the adjustable guide needle mechanism is connected to the adjusting knob, and the adjusting knob is rotated to drive a guide needle to stretch or retreat. By means of the design of the adjustable guide needle mechanism, the implantation length of the guide needle can be adjusted during production, and then the guide needle is configured with the corresponding emitter mechanism and is suitable for different users with various skin thicknesses; the production line and instrument input is reduced, and the implantation length can be changed adaptively during production to better satisfy customer requirements; though the cooperation of the adjustable guide needle mechanism and the adjusting knob, the guide needle can be drive to move upward or downward only by slightly rotating the adjustable guide needle mechanism or the adjusting knob, operation is easy, users can adjust the implantation length of the guide needle by themselves according to the measurement thickness of their skin, and discomfort of users caused by excessive implantation of the needle is reduced.

Further, a sliding slope is formed on the needle withdrawing member, the trigger module is provided with a needle withdrawing shell clamping buckle, which is matched with the sliding slope to limit the needle withdrawing member when the trigger module is locked.

Further, the adjusting knob comprises an index dial, a limiting step for preventing the adjusting knob from being separated from the outer shell, a connecting terminal connected to the adjustable guide needle mechanism, and an adjusting limit block is rotatably matched with an adjusting knob stop block of the adjusting knob connecting terminal, and every time the adjusting knob rotates by one circle, the guide needle stretches or retreats by 2-8 mm. By adjusting the stretching or retreating length of the guide needle through the adjusting knob, the implantation depth of the guide needle can be adjusted conveniently and quickly to ensure that the sensor is accurately implanted to a target depth to guarantee the accuracy of subcutaneous monitoring data obtained later, the structure is simple, operation is easy, and the stretching or retreating length of the guide needle can be visually displayed by the index dial.

Further, the needle withdrawing shell clamping buckle is used for clamping or releasing the needle withdrawing member, and when moving from the upper portion to the lower portion of the inner shell, the needle withdrawing shell clamping buckle releases the needle withdrawing member.

Further, the adjustable guide needle mechanism comprises a movable needle seat connected to the adjusting knob, a fixed needle seat rotatably connected to the movable needle seat, and the guide needle, and when the movable needle seat is rotated in a circumferential direction, the guide needle is driven to move upward and downward.

Further, an internal thread is formed in the movable needle seat, an external thread is formed on the guide needle, and a fixed needle seat cavity is formed in the fixed needle seat.

Further, connecting pins are formed on the movable needle seat or the fixed needle seat or are formed on the movable needle seat and the fixed needle seat to limit the movable needle seat and the fixed needle seat in an axial direction.

Further, a notch is formed in an outer wall of the fixed needle seat, and a three-jaw clip to be clamped in the notch is formed on the needle withdrawing member.

The invention has the following beneficial effects: the inner shell comprises the upper portion and the lower portion, which have different inner diameters, such that the trigger module can be locked and can be switched from the locked state to an unlocked state easily, that is, the needle aid device is easier to trigger, and the trigger module and the inner shell can cooperatively move more stably; the trigger module is easier to unlock by means of the trigger button outside the outer shell, and the whole needle implantation process is steadier; by means of the design of the adjustable guide needle mechanism, the implantation length of the guide needle can be adjusted during production, and then the guide needle is configured with the corresponding emitter mechanism and is suitable for different users with various skin thicknesses; the production line and instrument input is reduced, and the implantation length can be changed adaptively during production to better satisfy customer requirements; though the cooperation of the adjustable guide needle mechanism and the adjusting knob, the guide needle can be drive to move upward or downward only by slightly rotating the adjustable guide needle mechanism or the adjusting knob, operation is easy, users can adjust the implantation length of the guide needle by themselves according to the measurement thickness of their skin, and discomfort of users caused by excessive implantation of the needle is reduced; the implantation depth of the guide needle can be adjusted conveniently and quickly to ensure that the sensor is accurately implanted to a target depth to guarantee the accuracy of subcutaneous monitoring data obtained later, the structure is simple, operation is easy, and the stretching or retreating length of the guide needle can be visually displayed by the index dial; by means of the design of ribs and grooves in the needle aid device, the whole needle implantation process is steadier, user experience is improved, the structure is simpler, and the manufacturing complex and production cost are reduced; and the design of the lock bracket and the trigger button avoids mis-trigger of users, thus improving the safety of the needle aid device in use.

### DESCRIPTION OF THE DRAWINGS

FIG. 1 is an exploded structural view of the invention.

FIG. 2 is a perspective view of an adjusting knob according to the invention.

FIG. 3 is a top view and a front view of the adjusting knob according to the invention.

FIG. 4 is a first schematic diagram of an outer shell according to the invention.

FIG. 5 is a second schematic diagram of the outer shell according to the invention.

FIG. 6 is structural diagram of running fit between the outer shell and the adjusting knob according to the invention.

FIG. 7 is a perspective view of an inner shell according to the invention.

FIG. 8 is a partial sectional view of the inner shell according to the invention.

FIG. 9 is a perspective view of a trigger button according to the invention.

FIG. 10 is a perspective view of a lock bracket according to the invention.

FIG. 11 is a perspective view of a needle withdrawing member according to the invention.

FIG. 12 is a partial sectional view of the needle withdrawing member according to the invention.

FIG. 13 is a perspective view of an adjustable guide needle mechanism according to the invention.

FIG. 14 is a first perspective view of a trigger module according to the invention.

FIG. 15 is a second perspective view of a trigger module according to the invention.

FIG. 16 is an internal sectional view of the operating process according to the invention (the trigger button is pressed to unlock the trigger module).

FIG. 17 is an internal sectional view of the operating process according to the invention (the trigger module moves downward).

FIG. 18 is an internal sectional view of the operating process according to the invention (the trigger module releases an emitter mechanism).

FIG. 19 is a schematic diagram of the rotation of the adjusting knob during use according to the invention.

FIG. 20 is a schematic diagram of unlocking the trigger button by the lock bracket during use according to the invention.

FIG. 21 is a schematic diagram of placing the needle aid device in a desired implanting position and pressing the trigger button during use according to the invention.

FIG. 22 is an operating diagram of the emitter mechanism attached to the skin surface according to the invention.

FIG. 23 is a sectional view of an adjustable guide needle mechanism according to the invention.

FIG. 24 is an operating diagram of the adjustable guide needle mechanism according to the invention.

FIG. 25 is a partial sectional view of the adjustable guide needle mechanism according to the invention.

FIG. 26 is a perspective view of the emitter mechanism according to the invention.

### DETAILED DESCRIPTION OF THE EMBODIMENTS

To allow those skilled in the art to gain a better understanding of the technical solutions of the invention, the technical solutions of the embodiments of the invention will be clearly and completely described below in conjunction with the accompanying drawings of these embodiments. Obviously, the embodiments in the following description are merely illustrative ones, and are not all possible ones of the invention. All other embodiments obtained by those ordinarily skilled in the art according to the following ones without creative labor should also fall within the protection scope of the invention.

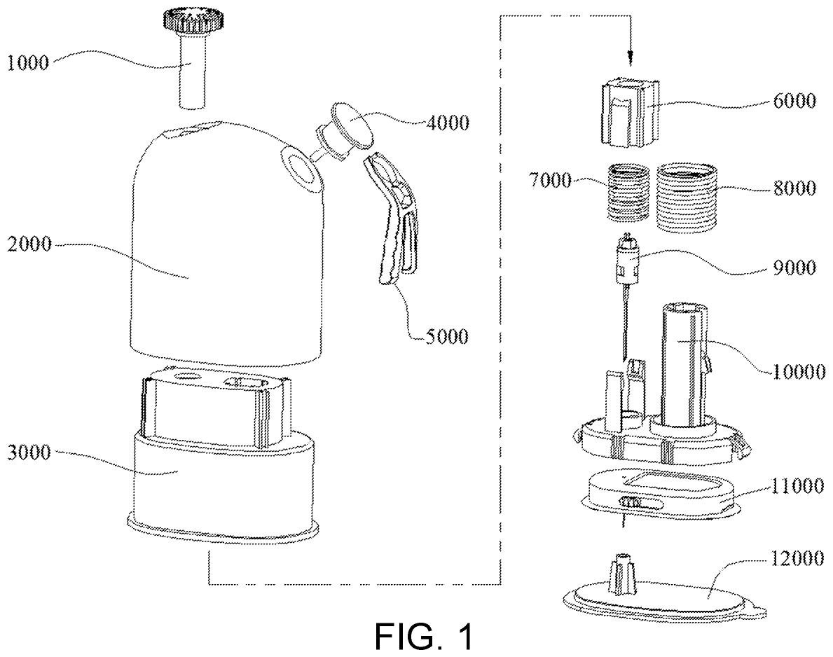

As shown in FIG. 1, a needle aid device comprises an adjusting knob 1000, an outer shell 2000, an inner shell 3000, a trigger button 4000, a lock bracket 5000, a trigger module 10000, an emitter mechanism 11000, a bottom cover 12000, a trigger spring 8000, a needle withdrawing spring 7000, a needle withdrawing member 6000, and a guide needle mechanism 9000. The inner shell 3000 is arranged in the outer shell 2000, an outer shell connecting terminal 2003 is arranged in the outer shell and connected to the inner shell 3000, the inner shell 3000 comprises an upper portion 3009 and a lower portion 3010, an inner diameter of the lower portion 3010 is greater than that of the upper portion 3009, and a trigger shell passage 3003 penetrating through the upper portion 3009 and the lower portion 3010 is formed in the inner shell 3000.

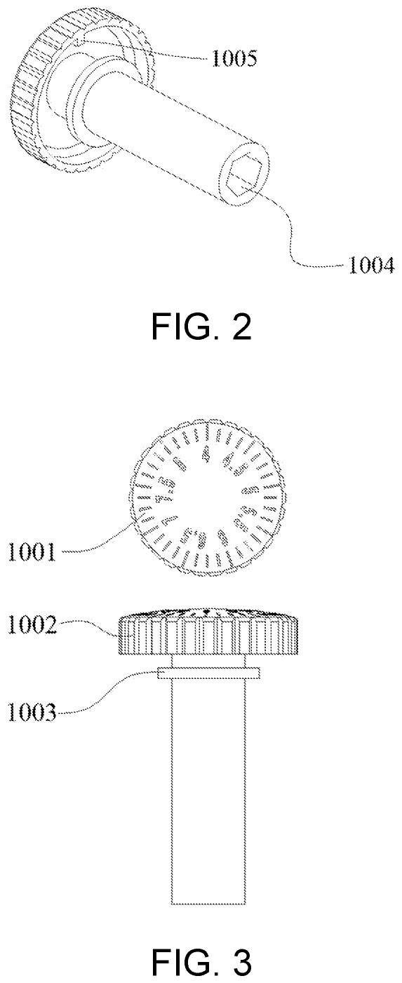

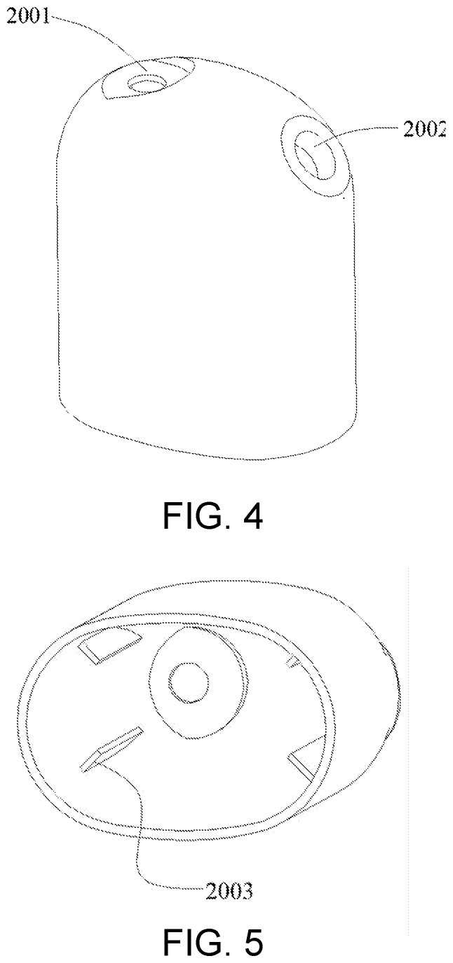

As shown in FIG. 2-FIG. 6, the outer shell 2000 comprises an adjusting knob connecting terminal 2001, a trigger button connecting terminal 2002, an outer shell connecting terminal 2003 and an adjusting knob stop block 2004. The adjusting knob connecting terminal 2001 is connected to a limiting step 1003 of the adjusting knob 1000, the trigger button connecting terminal 2002 is connected to the trigger button 4000, and the outer shell connecting terminal 2003 is matched and connected with an inner shell connecting terminal 3001 of the inner shell 3000, such that the outer shell 2000 and the inner shell 3000 are assembled; and the adjusting knob stop block 2004 is rotatably matched with an adjusting limit block 1005 to ensure that the adjusting knob 1000 can be clockwise or anticlockwise rotated within 360° and cannot be clockwise or anticlockwise rotated infinitely.

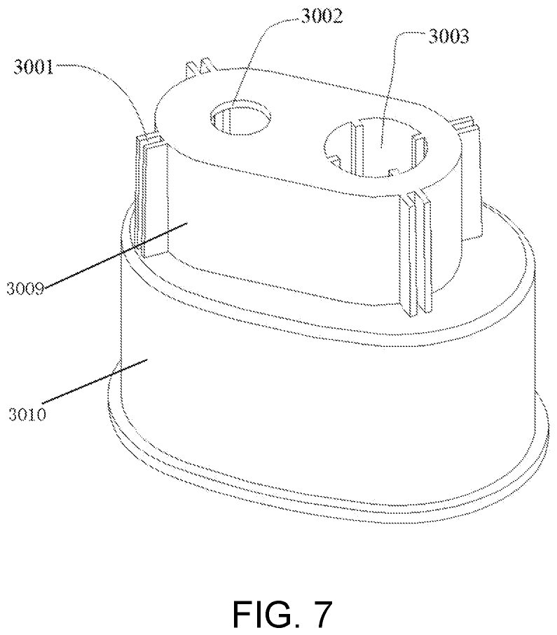

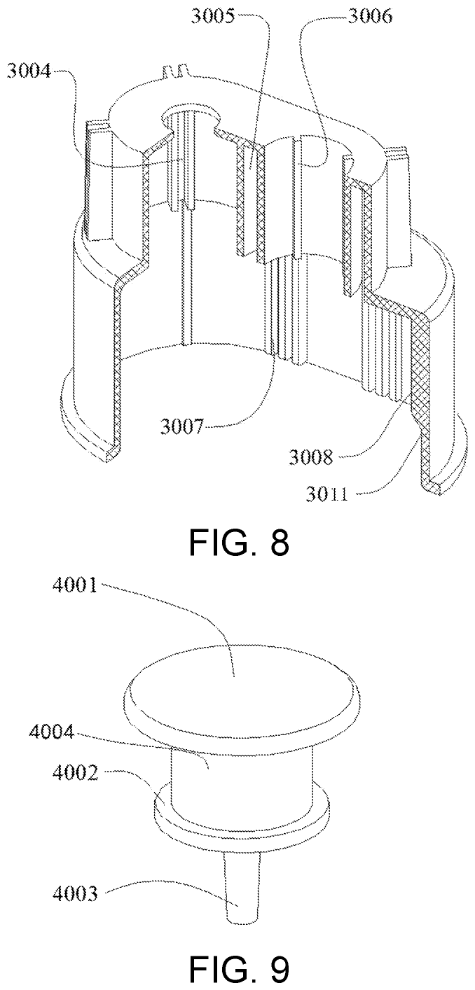

As shown in FIG. 7-FIG. 8, the trigger module 10000 is partially located in the trigger shell passage 3003 and is connected to the trigger shell passage 3003 through trigger shell guide ribs 3006 and trigger shell guide grooves 3007; the trigger module 10000 can move upward and downward along the trigger shell passage 3003 and is used for clamping or releasing the emitter mechanism 11000.

In an initial state, the trigger module 10000 is locked on the upper portion 3009, and specifically, the trigger module 1000 is locked at an opening of the trigger shell passage 3003 of the upper portion 3009.

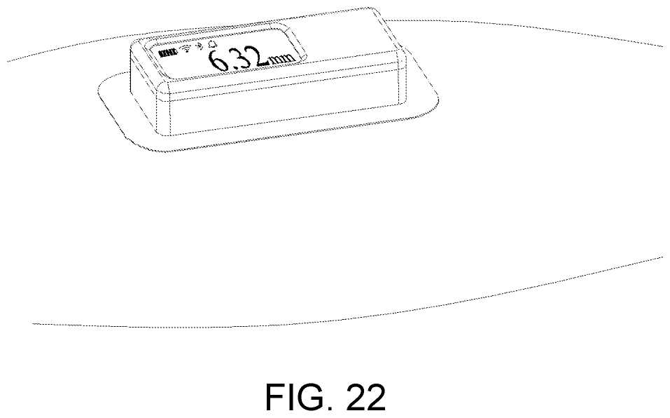

When unlocked, the trigger module 10000 is able to drive the emitter mechanism 11000 to move downward, clamping jaw stop ribs 3008 of the inner shell 3000 release the trigger module 10000 until the emitter mechanism 11000 moves to be separated from the trigger module 10000, and at this moment, the emitter mechanism 11000 can be attached to human skin, as shown in FIG. 22.

Specifically, as shown in FIG. 14, elastic clamping jaws 10004 are formed on the trigger module 10000, and are matched and connected with the emitter mechanism 11000. A thin-walled portion 3011 is formed on the lower portion 3010 of the inner shell 3000, and when the elastic clamping jaws 10004 move downward to the thin-walled part 3011, the thin-walled part 3011 release the elastic clamping jaws 10004, and thus the emitter mechanism 11000 is released.

To realize automatic trigger of the trigger module 10000, a trigger spring receiving groove 10005 for receiving the trigger spring 8000 is formed in the trigger module 10000, one end of the trigger spring 8000 abuts against the trigger module 10000, the other end of the trigger spring 8000 abuts against the inner shell 3000, and a trigger spring receiving groove 3005 is formed in the inner shell 3000.

As shown in FIG. 14, the trigger module 10000 further comprises a trigger buckle 10007. When the trigger module 10000 is locked, the trigger buckle 10007 makes contact with the trigger shell passage 3003 of the inner shell 3000, and specifically, the trigger buckle 10007 is clamped at the opening of the trigger shell passage 3003. That is, when the trigger module 10000 is not triggered, the trigger buckle 10007 clamps the edge of a top surface of the trigger shell passage 3003, such that the trigger module 10000 and the inner shell 3000 are locked together.

To unlock the trigger module 10000, the trigger button 4000 is connected to the outer shell 2000, and specifically, the trigger button 4000 is connected to the trigger button connecting terminal 2002 of the outer shell 2000. As shown in FIG. 9, the trigger button 4000 comprises a trigger surface 4001, a limiting step 4002, a trigger bar 4003 and a stop pillar 4004. The trigger surface 4001 is to be pressed by users, protrusions are arranged on the trigger surface 4001, such that users can press and recognize the trigger button 4000 conveniently; the trigger surface 4001 and the limiting step 4002 are located at two ends of the trigger button connecting terminal 2002, the trigger bar 4003 abuts against the trigger module 10000, and the trigger surface 4001 can be pressed to enable the trigger bar 4003 to abut against the trigger module 10000.

The trigger buckle 10007 is elastic, so when an external force is applied to the trigger buckle 10007, the trigger bar 4003 will push the trigger buckle 10007 to move away from the edge of the top surface of the trigger shell passage 3003, that is, the trigger bar 4003 is separated from the top surface of the inner shell 3000, at this moment, the trigger module 10000 will be unlocked, and the needle aid device will be triggered.

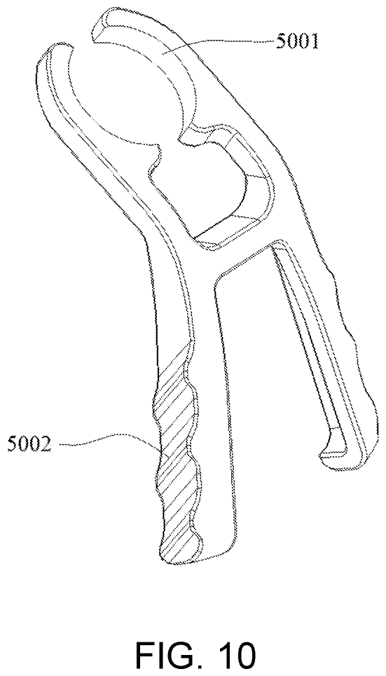

As shown in FIG. 10, in order to prevent the trigger button 4000 from moving freely, the trigger module 10000 further comprises the lock bracket 5000, the lock bracket 5000 comprises a clamp 5001 and a collet 5002. The clamp 5001 is located at a lower end of the trigger surface 4001 and an upper end of the trigger button connecting terminal 2002 and is used for locking the trigger button 4000, such that the trigger button 4000 will not be mistakenly triggered by users; lugs may be arranged on an inner of the clamp 5001, grooves may be formed in the stop pillar 4004, and through the cooperation between the lugs of the clamp 5001 and the grooves of the stop pillar 4004, the trigger button 4000 can be locked more efficiently; the collet 5002 is U-shaped, V-shaped or in other shapes beneficial to operation, such that users can press the collet 5002 with a single hand to open the clamp 5001 to unlock the trigger button 4000, and at this moment, users can press the trigger button 4000 to trigger the trigger module 10000.

To realize stable movement of the trigger module 10000 and the inner shell 3000, trigger shell guide ribs 3006 are arranged in the trigger shell passage 3003, the trigger module 10000 is provided with guide grooves 10006, and the guide grooves 10006 are matched with the trigger shell guide ribs 3006 to limit the movement of the trigger module 10000 in the axial direction, such that the implantation process is steadier.

Trigger shell guide grooves 3007 are formed in an inner wall of the inner shell 3000, the trigger module 10000 is provided with bottom guide grooves 10003, and the bottom guide grooves 10003 are matched with the trigger shell guide grooves 3007 to limit the movement of the trigger module 10000 in the axial direction, and the trigger process is steadier.

A guide needle passage is formed in the inner shell 3000, the needle withdrawing member 6000 for clamping or releasing the guide needle mechanism 9000 is mounted in the guide needle passage, the guide needle mechanism 9000 comprises a guide needle 9001, and the guide needle 9001 has a cavity for receiving a sensor.

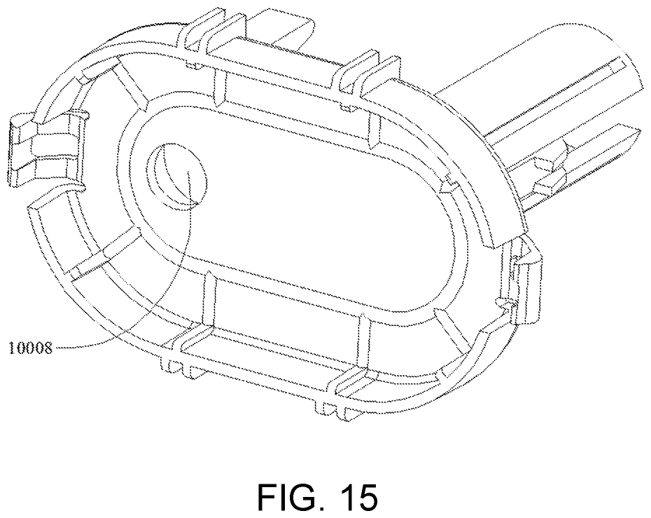

As shown in FIG. 15, the trigger module 10000 further comprises a needle passage 10008 allowing the guide needle to penetrate through.

The guide needle mechanism may be an existing product, or an adjustable guide needle mechanism 9000 which can adjust the stretching or retreating length of the guide needle.

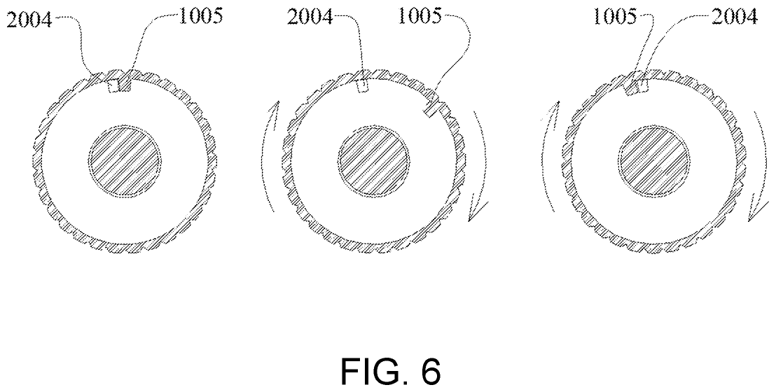

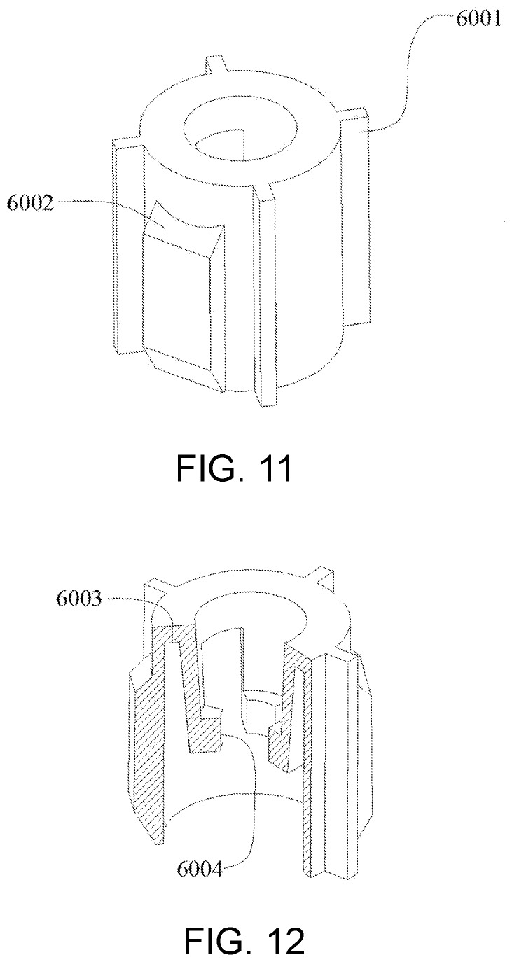

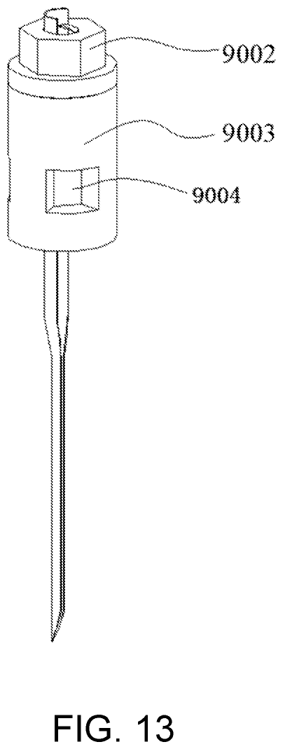

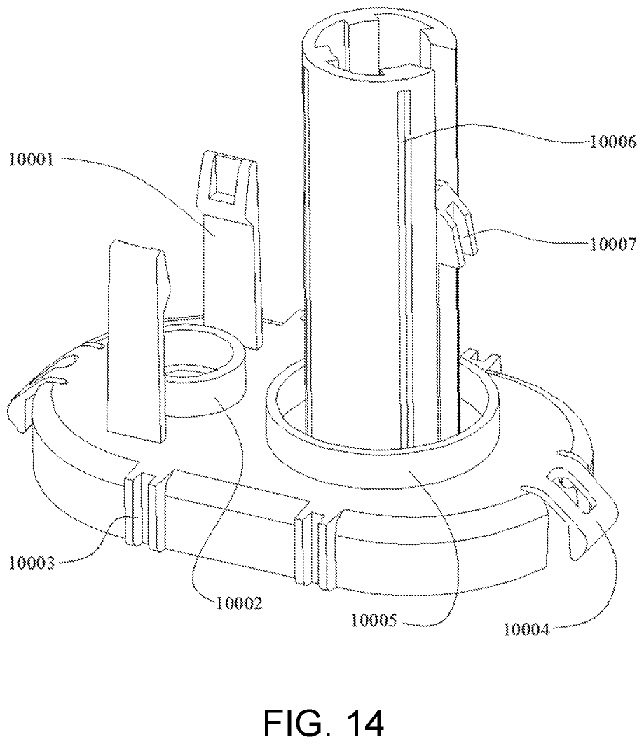

As shown in FIG. 13, the guide needle mechanism is an adjustable guide needle mechanism 9000 which can adjust the stretching or retreating length of the guide needle. In this case, as shown in FIG. 2, FIG. 3 and FIG. 6, an adjusting knob passage 3002 is formed in the inner shell 3000, and the adjusting knob 1000 is connected to the adjusting knob passage 3002. As shown in FIG. 11-FIG. 12, the needle withdrawing member 6000 comprises guide ribs 6001, a sliding slope 6002, a needle withdrawing spring receiving groove 6003 and a three-jaw clip 6004, the guide ribs 6001 are matched with needle withdrawing shell guide grooves 3004, the needle withdrawing member 6000 is able to slide upward and downward in the needle withdrawing shell guide grooves 3004 to realize stable needle implantation and withdrawal, the needle withdrawing spring and the trigger spring are not located on the same axis, such that radial movement along a single axis is reduced, and the situation that the needle withdrawing member 6000 will not get stuck during the movement process; the sliding slope 6002 is in contact with the trigger module 10000, specifically, a needle withdrawing shell clamping buckle 10001 is arranged on the trigger module 10000 and is matched with the sliding slope 6002 to fix and limit the needle withdrawing member 6000 in a non-triggered state; the needle withdrawing spring 7000 is received in the needle withdrawing spring receiving groove 6003 and is in a compressed state initially; and the three-jaw clip 6004 is matched with the adjustable guide needle mechanism 9000.

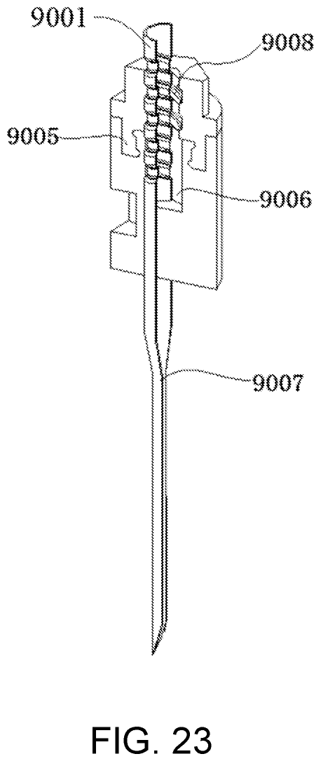

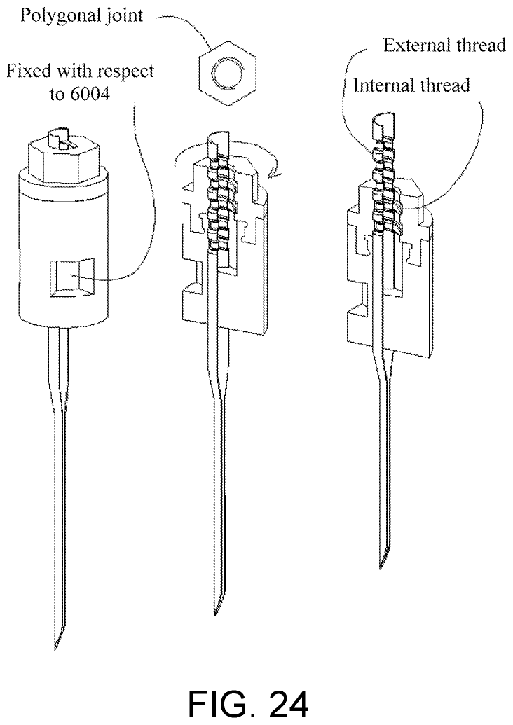

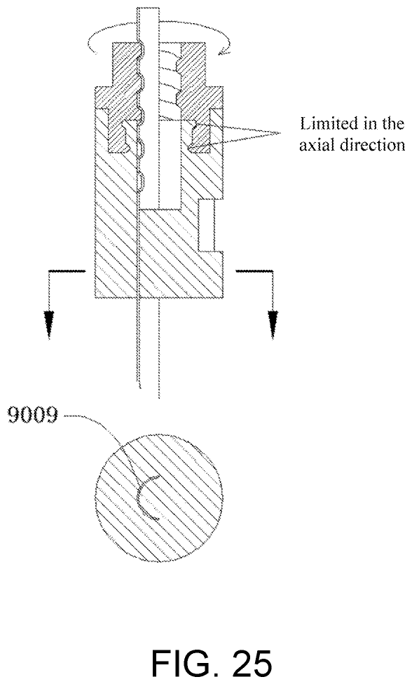

As shown in FIG. 13, and FIG. 23-FIG. 25, the adjustable guide needle mechanism 9000 comprises a guide needle 9001, a movable needle seat 9002 and a fixed needle seat 9003, wherein the guide needle 9001 is a non-sealed guide needle, an external thread is arranged at an upper end of the guide needle 9001, the middle of the guide needle 9001 is a smooth transition surface, and a lower end of the guide needle 9001 is an implantation end, which has a smooth surface and can receive a sensor 11003, that is, a cavity allowing the sensor 11003 of the emitter mechanism 11000 to stretch therein is formed in the lower end of the guide needle 9001. The movable needle seat 9002 comprises a connecting pin 9005 and an internal thread 9008, a clamping groove is formed in an end of the fixed needle seat 9003, and the connecting pin 9005 stretches into the clamping groove in the fixed needle seat 9003, such that the movable needle seat 9002 can rotate in the fixed needle seat 9003; or, a connecting pin 9005 is arranged on the fixed needle seat 9003, a clamping groove is formed in an end of the movable needle seat 9002, and the connecting pin 9005 stretches into the clamping groove in the movable needle seat 9002, such that the movable needle seat 9002 can rotate in the fixed needle seat 9003; or, connecting pins 9005 are arranged on the movable needle seat 9002 and the fixed needle seat 9003, and clamping grooves are formed in an end of the movable needle seat 9002 and an end of the fixed needle seat 9003. In this embodiment, clamping grooves are formed in an end of the movable needle seat 9002 and an end of the fixed needle seat 9003, and connecting pins 9005 are formed on the movable needle seat 9002 and the fixed needle seat 9003, such that the movable needle seat 9002 and the fixed needle seat 9003 are limited in the axial direction. In this embodiment, the connecting pin 9005 is a circular arc protrusion.

The movable needle seat 9002 is matched and connected with a connecting terminal 1004, the movable needle seat 9002 and the connecting terminal 1004 are circular or polygonal structures that can realize interference fit, the movable needle seat 9002 and the connecting terminal 1004 are matched with each other and are elastic, such that they can be disconnected when an external force is applied to the movable needle seat 9002, and the internal thread 9008 is matched with an external thread at an upper end of the guide needle 9001; a notch 9004 is formed in an outer surface of the fixed needle seat 9003, a fixed needle seat cavity 9006 and a middle section receiving groove 9009 of the guide needle 9001 are formed in the fixed needle seat 9003, and the notch 9004 is matched with the three-jaw clip 6004 of the needle withdrawing member to clamp the adjustable guide needle mechanism 9000; when the movable needle seat 9002 is rotated clockwise or anticlockwise, the guide needle 9001 threadedly connected with the movable needle seat 9002 will move upward or downward in the movable needle seat cavity 9006, such that the length of the implanted part of the guide needle is adjusted; the guide needle 9001 middle section receiving groove 9009 is semicircular or in other shapes matched with the cross-section of the middle section of the guide needle 9001, and only receives the middle section of the guide needle 9001 to limit the minimum length and maximum length of the guide needle 9001, such that the guide needle 9001 can be implanted more stably.

The adjusting knob 1000 can be rotated to drive the guide needle 9001 to stretch or retreat. Specifically, as shown in FIG. 2 and FIG. 3, the adjusting knob 1000 comprises an index dial 1001, a toothed cylindrical surface 1002, a limiting step 1003, a connecting terminal 1004 and an adjusting limit block 1005, wherein scale marks are arranged on the index dial 1001, and every time the adjusting knob 1000 is rotated by one circle, the guide needle 9001 stretches or retreats by 2-8 mm, preferably 4 mm; the initial value of the adjusting knob of the needle aid device is 4 mm, and the range of the scale marks on the index dial 1001 is 4 mm-8 mm; the toothed cylindrical surface 1002 can increase the friction between the adjusting knob 1000 and users, such that users can use the needle aid device conveniently; the limiting step 1003 is connected to the outer shell 2000, the index dial 10001 is connected to the outer shell 2000, and the limiting step 1003 and the index dial 1001 limit axial movement of the adjusting knob 1000 to prevent the adjusting knob 1000 from being separated from the outer shell 2000; the connecting terminal 1004 is connected to the adjustable guide needle mechanism 9000, and is made of a material with high friction, such that the connecting terminal 1004 will not rotate with respect to the adjustable guide needle mechanism 9000 unless a certain external force is applied to the connecting terminal 1004; and the adjusting limit block 1005 is rotatably matched with the adjustable knob stop block 2004, such that the adjusting knob 1000 can be rotated only within ±360°.

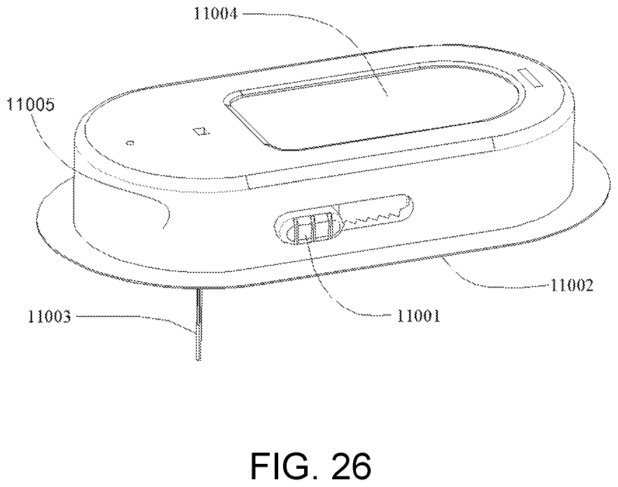

As shown in FIG. 26, the emitter mechanism 11000 comprises an adjusting module 11001, an adhesive paper 11002, a sensor 11003, a visible window 11004, and a housing 11005. The adjusting module 11001 is used for controlling and performing length adjustment of the sensor 11003; the adhesive paper 11002 is used for adhering the emitter mechanism 11000 to the surface of a human body; the sensor 11003 is used for detecting a blood glucose signal of the human body and supplying power; and the visible window 11004 is used for displaying the implantation depth of the sensor 11003 in human skin, the blood glucose level, the blood glucose monitoring curve, the electric quantity, WiFi/Bluetooth connection information, and other information.

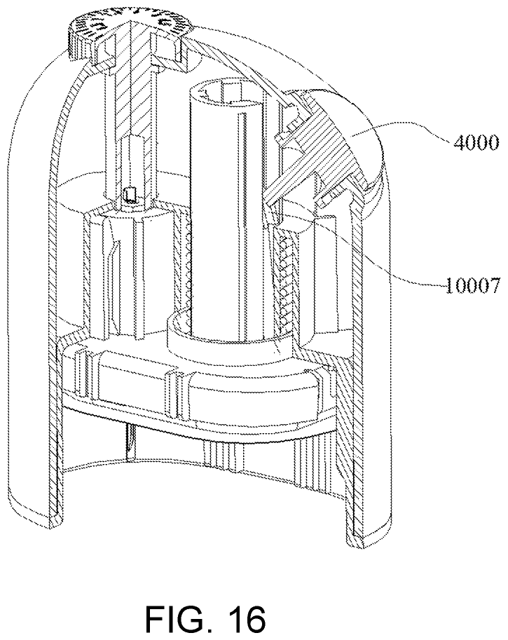

The operating process of the invention is as follows: as shown in FIG. 16, the trigger button 4000 is pressed, the trigger bar 4003 presses against the trigger buckle 10007, the trigger buckle 10007 moves away from the edge of the trigger shell passage 3003, and the needle aid device is triggered.

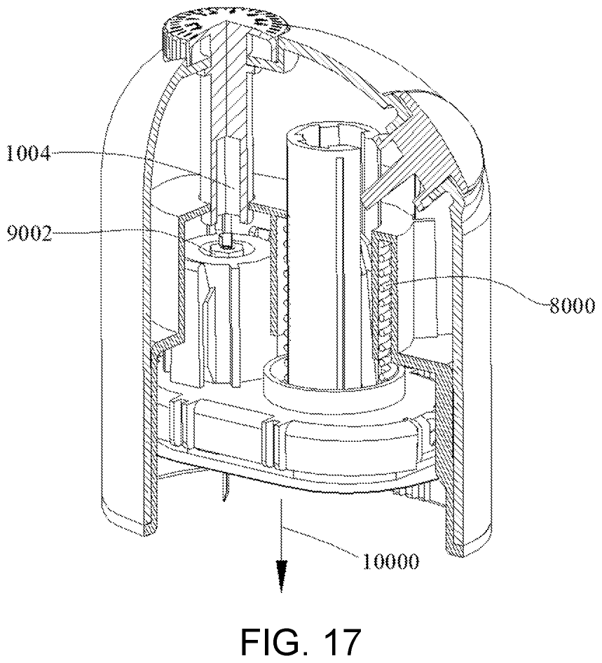

As shown in FIG. 17, the trigger buckle 10007 releases the inner shell 3000, the trigger module 10000 is driven by the elastic force of the trigger spring 8000 to move downward, and the guide needle mechanism 9000 is driven to move downward to be separated from the connecting terminal 1004.

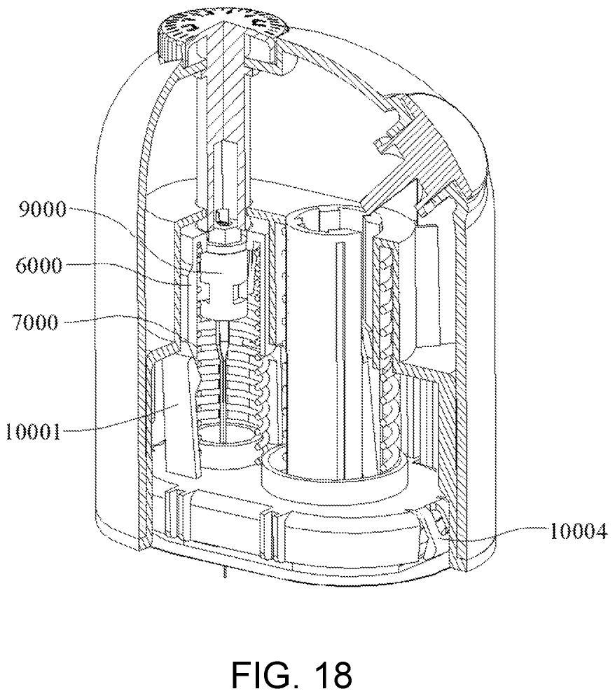

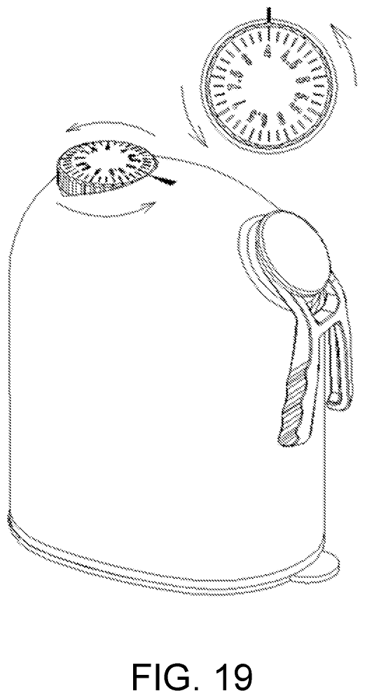

As shown in FIG. 18, when the elastic clamping jaws 10004 of the trigger module move to the thin-walled portion 3011 of the inner shell 3000, the needle withdrawing shell clamping buckle 10001 and the elastic clamping jaws 10004 of the trigger module release the emitter mechanism 11000 to allow the emitter mechanism 11000 to be adhered to the surface of human skin, the needle withdrawing shell clamping buckle 10001 releases the needle withdrawing member 6000, and at this moment, the needle withdrawing member 6000 is driven by the elastic force of the needle withdrawing spring 7000 to move upward to withdraw the needle, such that the sensor 11003 is smoothly implanted into the human body.

The needle aid device is used as follows: the skin thickness is detected by means of a thickness detection instrument, and on the basis of the initial implantation length, users adjust the index dial 1001 (clockwise/anticlockwise→increase/decrease) to adjust the implantation length of the guide needle 9001; the emitter mechanism 11000 automatically adjusts the implantation length of the sensor 110003 after receiving the skin thickness detected by the thickness detection instrument.

Or, the skin thickness is detected by means of a thickness detection instrument, and on the basis of the initial implantation length, and users adjust the index dial 1001 (clockwise/anticlockwise→increase/decrease) and manually adjust the implantation length of the sensor 110003 at the same time.

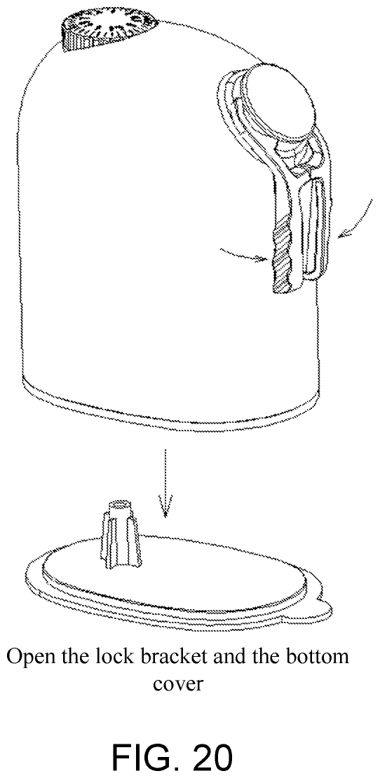

As shown in FIG. 20, users open the lock bracket and the bottom cover.

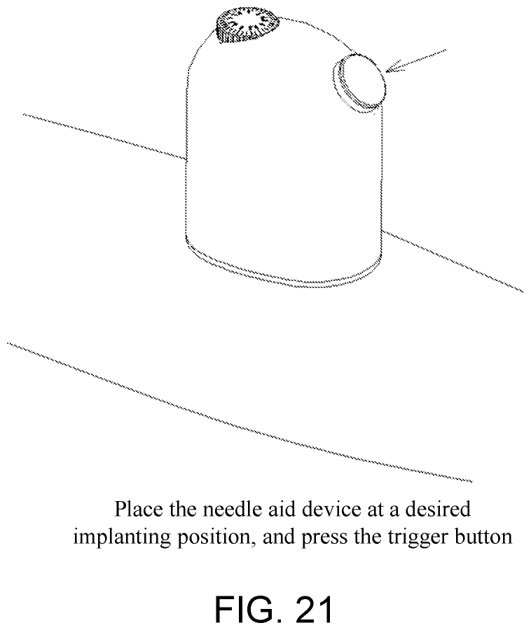

As shown in FIG. 21, users place the needle aid device at a desired implanting position and press the trigger button 4000 to trigger the needle aid device.

As shown in FIG. 22, the emitter mechanism 11000 is adhered to the skin surface and starts to operate, the visible window displays the implantation depth of the sensor in human skin, the blood glucose level, the blood glucose monitoring curve, the electric quantity, WiFi/Bluetooth connection information, and other information, and the visible window can display the implantation depth of the sensor in human skin, the electric quantity and WiFi/Bluetooth connection information when the sensor is adjusted.

The above specific embodiments are used for explaining the invention, and are not intended to limit the invention. Any modifications and changes of the invention made based on the spirit and protection scope of the claims should fall within the protection scope of the invention.

1. A needle aid device, comprising:
an outer shell;
an inner shell located in the outer shell and comprising an upper portion and a lower portion, a trigger shell passage penetrating through the upper portion and the lower portion being formed in the inner shell, and an inner diameter of the lower portion being greater than that of the upper portion; and
a trigger module capable of moving in the trigger shell passage and used for clamping or releasing an emitter mechanism;
in an initial state, the trigger module being locked on the upper portion; and
when unlocked, the trigger module drives the emitter mechanism to move downward until the emitter mechanism is separated from the trigger module.

2. The needle aid device according to claim 1, wherein elastic clamping jaws are formed on the trigger module and are used for clamping the emitter mechanism, a thin-walled portion is formed on the lower portion of the inner shell, and when moving downward to the thin-walled portion, the elastic clamping jaws release the emitter mechanism.

3. The needle aid device according to claim 1, wherein the trigger module is provided with a trigger buckle; when the trigger module is locked, the trigger buckle is clamped at an opening of the trigger shell passage; and when an external force is applied to the trigger button to push the trigger buckle away from a top surface of the inner shell, the trigger module is unlocked.

4. The needle aid device according to claim 1, wherein a trigger button and a trigger button connecting terminal are connected to the outer shell, the trigger button is movably connected to the trigger button connecting terminal, and an external force is applied to the trigger button to unlock the trigger module.

5. The needle aid device according to claim 4, wherein the trigger module further comprises a lock bracket, the lock bracket comprises a clamp and a collet, and the clamp clamps the trigger button to prevent the trigger button from moving with respect to the outer shell.

6. The needle aid device according to claim 1, wherein trigger shell guide ribs are arranged in the trigger shell passage, the trigger module is provided with guide grooves, and the trigger shell guide ribs are clamped in the guide grooves and allowed to move in the guide grooves to realize vertical movement of the trigger module.

7. The needle aid device according to claim 1, wherein trigger shell guide grooves are formed in an inner wall of the inner shell, the trigger module is provided with bottom guide grooves, and the trigger shell guide grooves are matched with the bottom guide grooves and allowed to move with respect to the bottom guide grooves to realize vertical movement of the trigger module.

8. The needle aid device according to claim 1, wherein the needle aid device further comprises a guide needle passage formed in the inner shell and a needle withdrawing member for clamping or releasing a guide needle mechanism, and a cavity allowing a sensor of the emitter mechanism to stretch therein is formed in a guide needle.

9. The needle aid device according to claim 1, wherein the needle aid device further comprises an adjusting knob passage formed in the inner shell, an adjusting knob, an adjusting knob connecting terminal, an adjustable guide needle mechanism, and a needle withdrawing member for clamping or releasing the adjustable guide needle mechanism; part of the adjusting knob stretches into the adjusting knob passage, the adjusting knob is rotatably connected to the adjusting knob connecting terminal, the adjustable guide needle mechanism is connected to the adjusting knob, and the adjusting knob is rotated to drive a guide needle to stretch or retreat.

10. The needle aid device according to claim 8, wherein a sliding slope is formed on the needle withdrawing member, the trigger module is provided with a needle withdrawing shell clamping buckle, which is matched with the sliding slope to limit the needle withdrawing member when the trigger module is locked.

11. The needle aid device according to claim 9, wherein the adjusting knob comprises an index dial, a limiting step for preventing the adjusting knob from being separated from the outer shell, a connecting terminal connected to the adjustable guide needle mechanism, and an adjusting limit block is rotatably matched with an adjusting knob stop block of the adjusting knob connecting terminal, and every time the adjusting knob rotates by one circle, the guide needle stretches or retreats by 2-8 mm.

12. The needle aid device according to claim 10, wherein the needle withdrawing shell clamping buckle is used for clamping or releasing the needle withdrawing member, and when moving from the upper portion to the lower portion of the inner shell, the needle withdrawing shell clamping buckle releases the needle withdrawing member.

13. The needle aid device according to claim 9, wherein the adjustable guide needle mechanism comprises a movable needle seat connected to the adjusting knob, a fixed needle seat rotatably connected to the movable needle seat, and the guide needle, and when the movable needle seat is rotated in a circumferential direction, the guide needle is driven to move upward and downward.

14. The needle aid device according to claim 13, wherein an internal thread is formed in the movable needle seat, an external thread is formed on the guide needle, and a fixed needle seat cavity is formed in the fixed needle seat.

15. The needle aid device according to claim 13, wherein connecting pins are formed on the movable needle seat or the fixed needle seat or are formed on the movable needle seat and the fixed needle seat to limit the movable needle seat and the fixed needle seat in an axial direction.

16. The needle aid device according to claim 13, wherein a notch is formed in an outer wall of the fixed needle seat, and a three-jaw clip to be clamped in the notch is formed on the needle withdrawing member.

17. The needle aid device according to claim 6, wherein trigger shell guide grooves are formed in an inner wall of the inner shell, the trigger module is provided with bottom guide grooves, and the trigger shell guide grooves are matched with the bottom guide grooves and allowed to move with respect to the bottom guide grooves to realize vertical movement of the trigger module.

18. The needle aid device according to claim 9, wherein a sliding slope is formed on the needle withdrawing member, the trigger module is provided with a needle withdrawing shell clamping buckle, which is matched with the sliding slope to limit the needle withdrawing member when the trigger module is locked.
